(19) United States
(12) Patent Application Publication
(10) Pub. No.: US 2017/0216531 A1
(43) Pub. Date: Aug. 3, 2017

(54) CRYOSYRINGE DESIGNED FOR STERILE LIQUID NITROGEN FREEZING, QUICK THAWING AND INJECTION OF CELLULAR SLURRIES

(71) Applicants: THE JOHNS HOPKINS UNIVERSITY, Baltimore, MD (US); U.S. Army Medical Research Material Command, Fort Detrick, MD (US)

(72) Inventors: Luis Andres GARZA, Baltimore, MD (US); Jon MEYERLE, Chevy Chase, MD (US); Sewon KANG, Phoenix, MD (US)

(21) Appl. No.: 15/500,684

(22) PCT Filed: Jul. 30, 2015
(86) PCT No.: PCT/US2015/042830

§ 371 Date: Jan. 31, 2017

Related U.S. Application Data
(60) Provisional application No. 62/031,187, filed on Jul. 31, 2014.

(51) Int. Cl. A61M 5/31

(52) U.S. Cl. CPC A61M 5/3134; A61M 5/3135; A61M 2202/09; A61M 2005/3131; A61M 2005/3125; A61M 2205/3633

(57) ABSTRACT

An embodiment in accordance with the present invention is directed to a device for use as a cryosyringe and a cryo-storage vial. The device includes a first end and a second end and has an outer wall defining a lumen between the first and second ends. Both the first end and the second end include threads configured for attaching a first cap to the first end and a second cap to the second end. The device can also include a syringe plunger adapter, such that the cryo-storage vial is convertible into a cryosyringe to ensure sterility of the contents. Similarly, a luer-lock adapter can be included to further convert the device into a cryosyringe. The device and its components are formed from a cryo-resistant material. The device can therefore withstand liquid nitrogen temperatures necessary to preserve cell viability and be injected without compromising sterility to dispense the cellular therapy.

### CROSS REFERENCE TO RELATED APPLICATIONS

This application claims the benefit of U.S. Provisional Patent Application No. 62/031,187 filed Jul. 31, 2014, which is incorporated by reference herein, in its entirety.

### GOVERNMENT SUPPORT

This invention was made with government support under A66001-10-1-4081 awarded by the Defense Advanced Research Products Agency and Investigational New Drug Support from the United States Army Medical Material Development Activity and under AR055666 and AR064297-02 awarded by the National Institutes of Health. The government has certain rights in the invention.

### FIELD OF THE INVENTION

The present invention relates generally to medical devices. More particularly, the present invention relates to a cryosyringe designed for sterile liquid nitrogen freezing, quick thawing and injection of cellular slurries.

### BACKGROUND OF THE INVENTION

Fill-and-finish is the last step of the manufacturing process, wherein pharmaceuticals, compounds, or cells are placed into a vial and the vial is sealed. Because of the quality standards necessary to ensure sterility of the contents, fill-and-finish can be a costly step in the manufacturing process. The sterility of the contents is of the utmost importance, because the contents may at some time be administered to a patient. One source of potential contamination is the transfer of the contents to a syringe for administering to the patient. After transfer to a syringe, costly steps to verify sterility often must be completed again.

Accordingly, there is a need in the art for a device to allow for conversion of the cryo-storage vial into a syringe for administering the contents to a patient.

### SUMMARY OF THE INVENTION

The foregoing needs are met, to a great extent, by the present invention which provides a device for storage and delivery of a sample including a tube formed from a cryo-resistant material having an outer wall defining an inner lumen. The tube also has a first end and a second end, wherein the first and second ends define openings. A first end cap is configured to couple with the first end of the tube. The first end of the tube is configured to couple with the first end cap, such that the first end cap seals the first opening of the tube. A second end cap is configured to couple with the second end of the tube. The second end of the tube is configured to couple with the second end cap, such that the second end cap seals the second opening of the tube. The tube, the first end cap, and the second end cap are all formed from a cryo-resistant material.

In accordance with an aspect of the present invention, the first and second ends of the tube are threaded. The first and second end caps are threaded, such that they couple to the first and second ends of the tube. The cryo-resistant material is a medical-grade polypropylene or other medical grade plastic. The device includes a syringe plunger adapter, and the device includes a luer-lock adapter. The device includes cryo-resistant gaskets positioned at the first end and the second end of the tube. The cryoresistant gaskets are configured to create a seal between the first and second ends and the first and second end caps. The tube includes gradations, and the gradations are formed by printing, pressing, etching, or engraving on the tube. The cryo-resistant material is configured to withstand liquid nitrogen temperatures. The device is configured to dispense its contents without a change of container. The cryo-resistant material is a plastic.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a side view of a device for use as a cryo-storage vial and a cryosyringe according to an embodiment of the present invention.

FIG. 2 illustrates an exploded view of a device for use as a cryo-syringe vial and a cryosyringe according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention is directed to a device for use as a cryosyringe and a cryo-storage vial. The device includes a first end and a second end and has an outer wall defining a lumen between the first and second ends. Both the first end and the second end include threads configured for attaching a first cap to the first end and a second cap to the second end. The device can also include a syringe plunger adapter, such that the cryo-storage vial is convertible into a cryosyringe to ensure sterility of the contents. Similarly, a luer-lock adapter can be included to further convert the device into a cryosyringe. The device and its components are formed from a cryo-resistant material. The device can therefore withstand liquid nitrogen temperatures necessary to preserve cell viability and be injected without compromising sterility to dispense the cellular therapy.

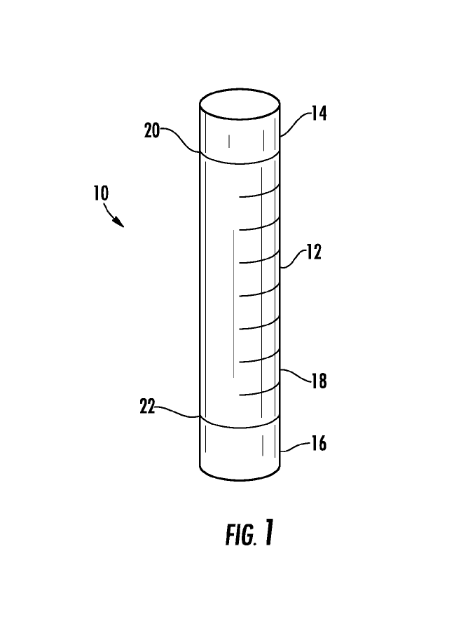

FIG. 1 illustrates a side view of a device for use as a cryo-storage vial and cryosyringe according to an embodiment of the present invention. The device 10 includes a tube 12, a first end cap 14, and a second end cap 16. The tube 12 includes an outer wall defining an inner lumen that extends from the first end 22 to the second end 24. The outer wall of tube 12 also preferably includes gradations that are printed, pressed, etched, or engraved into the tube 12. The gradations can also be produced on the vial in any other way known to or conceivable by one of skill in the art. The first end cap 14 and the second end cap 16 cover the openings to the inner lumen and keep the contents of the device inside. The device 10 including the end caps 14, 16 and the tube 12 are all formed from a cryo-resistant material such as a plastic. Preferably, the end caps 14, 16, and the tube 12 are formed from a material such as a medical grade polypropylene. The material preferably is resistant to liquid nitrogen freezing.

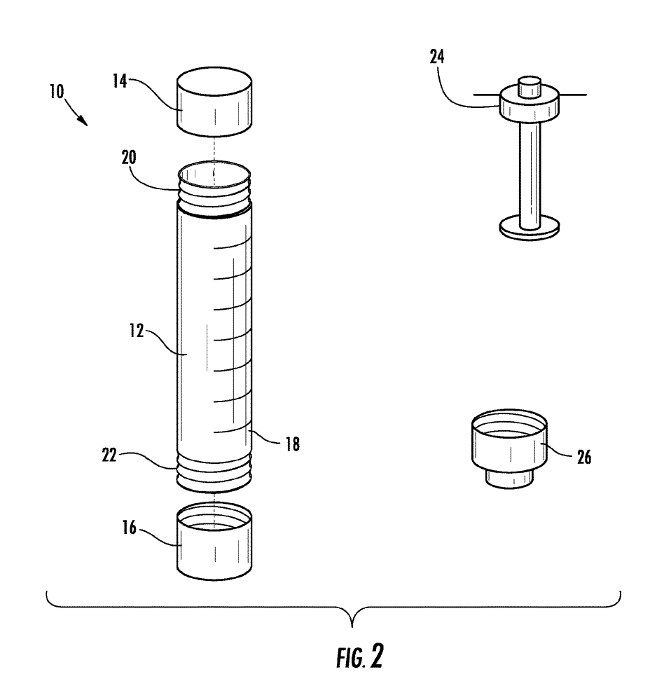

FIG. 2 illustrates an exploded view of a cryo-syringe vial according to an embodiment of the present invention. The device 10 includes tube 12, first end cap 14, and second end cap 16. The tube 12 includes the first end 20 and the second end 22, and the outer wall of the tube defines the interior lumen that extends from the first end 20 to the second end 22. The lumen also defines openings that the first end cap 14 and the second end cap 16 cover. The openings allow for filling and dispensing contents into and from the device. The tube 12 can also include gradations that are printed, pressed, etched, or engraved into the tube 12. The gradations can also be produced on the vial in any other way known to or conceivable by one of skill in the art. The tube 12 preferably includes threading for coupling the first and second end caps 14, 16 to the first and second ends 20, 22 of the tube. As illustrated in FIG. 2, the threading is external. However, the threading can be placed in any suitable manner known to or conceivable by one of skill in the art, such as internal threading. The end caps are threaded in a way that is complimentary to the threading of the tube, such that the caps can be secured to the tube, and such that the openings of the tube are sealed. The first and second end caps 14, 16 can also be secured in any other suitable fashion known to or conceivable by one of skill in the art, such as frictional fit and tab and groove.

FIG. 2 also illustrates additional components for the device including a plunger adapter 24 and a luer-lock adapter 26. The plunger adapter 24 and the luer-lock adapter 26 are used to convert the device 10 into a syringe from its vial state. The luer-lock adapter 26 allows for a needle to be coupled to the device 10 and the plunger 24 allows for the contents of the tube 12 to be expelled, while maintaining sterility. Preferably, the end caps 14, 16, and the tube are formed from a material such as a medical-grade polypropylene. The plunger adapter 24 and the luer-lock adapter 26 can also be formed from a cryo-resistant material such as a medical-grade polypropylene, or from any other suitable material known to one of skill in the art to survive liquid nitrogen freezing. The plunger adapter 24 and the luer-lock adapter 26 can be formed from non-cryo-resistant materials as well. Alternately, cryo-resistant and non-cryo-resistant plunger adapters and luer-lock adapters can be included with the device. The device can also include gaskets formed from a cryo-resistant material for placement at the first end 20 and the second end 22 to ensure that the first cap 14 and the second cap 16 fit the tube 12 tightly, without the caps 14, 16 being over-tightened.

The present invention minimizes the effort, cost, and labor of fill and finish for cellular therapies. The present invention also obviates the need to verify sterility after freezing of cells destined for cellular therapy, because the sterility of the cells is never compromised by changing containers. Generally, cells are washed and transferred to a new syringe for administration to the patient. The present invention eliminates the need for such a process.

The many features and advantages of the invention are apparent from the detailed specification, and thus, it is intended by the appended claims to cover all such features and advantages of the invention, which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device for storage and delivery of a sample comprising:
a tube formed from a cryo-resistant material having an outer wall defining an inner lumen, the tube also having a first end and a second end, wherein the first and second ends define openings, wherein the openings are in communication with the lumen;
a first end cap configured to couple with the first end of the tube, and wherein the first end of the tube is configured to couple with the first end cap, such that the first end cap seals the first opening of the tube;
a second end cap configured to couple with the second end of the tube, and wherein the second end of the tube is configured to couple with the second end cap, such that the second end cap seals the second opening of the tube; and
wherein the tube, the first end cap, and the second end cap are all formed from a cryo-resistant material.

2. The device of claim 1 wherein the first and second ends of the tube are threaded.

3. The device of claim 2 wherein the first and second end caps are threaded, such that they couple to the first and second ends of the tube.

4. The device of claim 1 wherein the cryo-resistant material is a medical-grade polypropylene.

5. The device of claim 1 wherein the device includes a syringe plunger adapter.

6. The device of claim 1 wherein the device includes a luer-lock adapter.

7. The device of claim 1 wherein the device includes a cryo-resistant gasket positioned at the first end of the tube.

8. The device of claim 7 wherein the cryo-resistant gasket is configured to create a seal between the first end and the first end cap.

9. The device of claim 1 wherein the device includes a cryo-resistant gasket positioned at the second end of the tube.

10. The device of claim 9 wherein the cryo-resistant gasket is configured to create a seal between the second end and the second end cap.

11. The device of claim 1 wherein the tube includes gradations.

12. The device of claim 11 wherein the gradations are formed by one selected from a group consisting of printing, pressing, etching, or engraving on the tube.

13. The device of claim 1 wherein the cryo-resistant material is configured to withstand liquid nitrogen temperatures.

14. The device of claim 1 wherein the device is configured to dispense its contents without a change of container.

15. The device of claim 1 wherein the cryo-resistant material is a plastic.
